(19) United States
(12) Patent Application Publication
(10) Pub. No.: US 2006/0188556 A1
(43) Pub. Date: Aug. 24, 2006

(54) Ultrasonically assisted dermal or transdermal delivery substance preparation

(76) Inventor: Bruce K. Redding, Broomal, PA (US)

Correspondence Address: PLEVY & HOWARD, P.C., P.O. BOX 226, FORT WASHINGTON, PA, 19034, US

(21) Appl. No.: 11/361,387

(22) Filed: Feb. 24, 2006

Related U.S. Application Data
(60) Provisional application No. 60/655,381, filed on Feb. 24, 2005.

(51) Int. Cl. A61K 38/28; A61N 1/30; A61F 13/02

(52) U.S. Cl. 424/448; 514/3; 604/20

(57) ABSTRACT

A method for improving transdermal delivery of an active ingredient in a formulation made in accordance with a dibasic sodium phosphate containing formula responsively to insonification thereof, including: reducing the amount of dibasic sodium phosphate in the formula to provide a reduced dibasic sodium phosphate formula; and, making a formulation in accordance with the reduced dibasic sodium phosphate formula.

### RELATED APPLICATION

This application claims priority of U.S. Patent Application Ser. No. 60/655,381, entitled UTRASONIC DRUG DELIVERY SYSTEM AND METHOD FOR THE MODIFICATION OF PHARMACEUTICAL PREPARATIONS, filed Feb. 24, 2005, the entire disclosure of which is hereby incorporated by reference as if being set forth in its entirety herein.

### FIELD OF THE INVENTION

The present invention relates generally to substance delivery methods, and more particularly to methods suitable for enhancing dermal and transdermal substance delivery.

### BACKGROUND OF THE INVENTION

Generally, transdermal medicinal compound, or drug, delivery systems employ a medicated patch, which is affixed to the skin of a patient. The patch allows the medicinal compound to be absorbed through the skin layers and into the patient's blood stream. Transdermal drug delivery generally mitigates the pain associated with injections and intravenous administration, as well as the risk of infection associated with these techniques. Transdermal drug delivery also avoids gastrointestinal metabolism of administered drugs, mitigates liver metabolization thereof, and provides a sustained release of the administered drug. Transdermal drug delivery also enhances patient compliance with a drug regimen, because of the relative ease of administration and the sustained release of the drug.

Many drugs, however, are not well suited for administration via conventional transdermal drug delivery systems. For example, some substances are absorbed with difficulty through the skin due to molecular size or bio-adhesion properties. In these cases, when transdermal drug delivery is attempted, the drug may pool on the outer surface of the skin, not permeating through the skin into the blood stream. An example of such a drug is insulin, which has been found difficult to administer via conventional transdermal drug delivery systems.

Consistently, conventional transdermal drug delivery methods have been found suitable only for low molecular weight medications−such as nitroglycerin for alleviating angina, nicotine for smoking cessation regimens, and estradiol for estrogen replacement in post-menopausal women. Larger molecular medications such as insulin (a polypeptide for the treatment of diabetes), erythropoietin (used to treat severe anemia) and gamma-interferon (used to boost the immune systems cancer fighting ability) are all compounds not normally effective when used with conventional transdermal drug delivery methods.

One method of assisting transdermal drug delivery is by iontophoresis. Iontophoresis involves the application of an external electric field and topical delivery of an ionized form of drug or unionized drug carried with the water flux associated with ion transport (electro-osmosis). Iontophoresis has been proposed to increase the permeability of skin to drugs. While permeation enhancement with iontophoresis may be effective, control of drug delivery and irreversible skin damage are problems associated with the technique.

Ultrasound has also been suggested to enhance permeability of the skin. Ultrasonic emissions, or signals, can be generated by vibrating a piezoelectric crystal or other electromechanical element, such as by passing an alternating current there through. The use of ultrasound to increase the permeability of the skin to drug molecules is sometimes referred to as sonophoresis or phonophoresis. However, while the use of ultrasound for drug delivery has been generally suggested, results have been largely disappointing—in that enhancement of skin permeability has been relatively low. Further, it is believed that there is no consensus on the efficacy of ultrasound for increasing drug flux across the skin. While some studies report the success of sonophoresis, others have obtained negative results.

In view of the foregoing, safely enhancing drug transport across the skin is desirable.

### SUMMARY OF THE INVENTION

A method for improving transdermal delivery of an active ingredient in a formulation made in accordance with a dibasic sodium phosphate containing formula responsively to insonification thereof, including: reducing the amount of dibasic sodium phosphate in the formula to provide a reduced dibasic sodium phosphate formula; and, making a formulation in accordance with the reduced dibasic sodium phosphate formula.

### BRIEF DESCRIPTION OF THE FIGURES

Understanding of the present invention will be facilitated by consideration of the following detailed description of the preferred embodiments of the present invention taken in conjunction with the accompanying drawings, in which like numerals refer to like parts and in which:

FIG. 1 illustrates an absorbent pad incorporating patch suitable for use with ultrasonic signals to deliver a substance transdermally into a patient; and,

FIG. 2 illustrates a chart indicative of different transdermal delivery rates for different commercially available forms of insulin.

### DETAILED DESCRIPTION OF THE INVENTION

It is to be understood that the figures and descriptions of the present invention have been simplified to illustrate elements that are relevant for a clear understanding of the present invention, while eliminating, for purposes of clarity, many other elements found in typical pharmaceutical preparations, as wells as transdermal delivery methods and systems. However, because such elements are well known in the art, and because they do not facilitate a better understanding of the present invention, a discussion of such elements is not provided herein. The disclosure herein is directed to all such variations and modifications known to those skilled in the art.

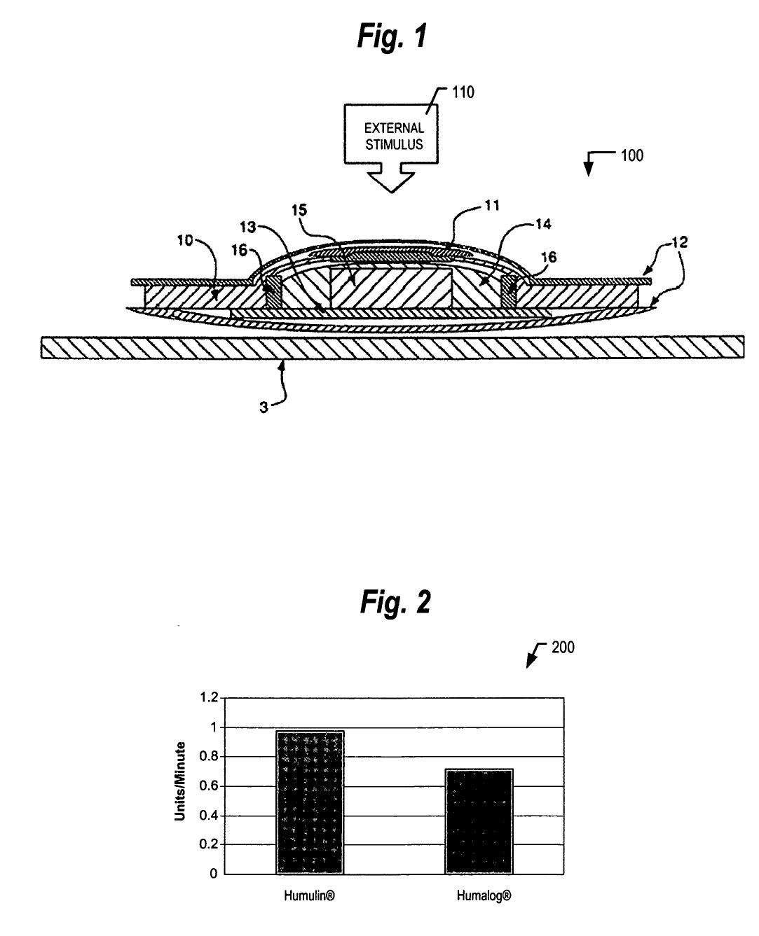

Applicants believe large molecular formulations can be effectively delivered responsively to ultrasound application or insonification. FIG. 1 illustrates a transdermal patch 100 suitable for use with ultrasonic signals, wherein the patch employs an absorbent pad. Patch 100 is constructed with a backbone or backing material 10 into which a section, or aperture, has been created. In the illustrated embodiment, the aperture accommodates a sonic membrane 11. A peel-away film 12 seals patch 300 until use. Peel-away film 12 may be constructed of any suitable material, including, but not limited to, UV-resistant, anti-static polyethylene film (50 micrometer thickness) available from Crystal-X Corp., of Sharon Hill, Pa. In the illustrated embodiment, and oppositely disposed from membrane 11, is a semi-permeable member 13. Member 13 may take the form of a membrane or film that comes into functional proximity with the skin of a user. For example, the patch may be adhered to the skin such that membrane 13 is in direct contact with the skin. In the interior of patch 100 is an absorbent pad 14 that holds a desired substance, e.g., drug or medicinal compound 15. In the illustrated embodiment, a gasket 16 is placed between backbone 10 and absorbent pad 14. Gasket 16 may be composed of any suitable material, such as, for example, synthetic rubber. Gasket 16 forms a reservoir or well over which absorbent pad 14 is placed. When patch 100 is pressed upon the skin, gasket 16 forms a barrier, which tends to restrict moisture and air from traveling under the patch and interfering with the ultrasonic signal intensity. Alternatively, a sealant compound, ultrasonic gel or other suitable material may be used for or in place of the gasket 16 to provide a sealing action around the borders of patch 100 to provide moisture protection, prevent leakage of substance or the drug from the patch and prevent air from entering under the patch. Of course, numerous changes to the components or construction of patch 100 may be made without departing from the spirit of the invention disclosed herein.

Regardless, an external stimulus 110, such as a source of ultrasonic signals, transmits emissions or signals into patch 100, and pad 14, through membrane 11. Substance 15, contained within the absorbent pad 14, is released in response to the impinging signals. The substance then passes through semi-permeable membrane 13 and is deposited on, in or through the surface of the patient's skin 3. While patch 100 has been described for use with ultrasound 110, other forms of external stimulus may be used in addition or in lieu of ultrasound. For example, iontophoresis, heat therapy, radio waves, magnetic transmission lasers, microwave signals, and/or electric currents applied to the skin may be used as the external stimulus. For example, ultrasonic signals may be used together with iontophoresis, or ultrasound may be used as a pre-treatment to iontophoresis, or vice-a-versa.

The terms “drug”, “medicinal compound”, “pharmaceutically active compound” and “pharmaceutical preparation”, as used herein, should be understood to be used in a non-limiting manner and for purposes of explanation only, as the present invention is suitable for delivering many substances or formulations to a patient. Such substances typically include one or more active ingredients and an excipient. “Excipient”, as used herein, generally refers to a substance used as a diluent or vehicle in a drug for the one or more active ingredients. Excipients are generally inert. Similarly, transdermally is used herein in a non-limiting manner, and includes intra-dermally (e.g., dermal delivery) and transmucosally as well, for example.

A pharmaceutical preparation to be transdermally delivered is preferably in a liquid form. A liquid excipient is used as the carrier for the active substance. For example, a substance to be delivered transdermally may typically consist of an active substance suspended within a liquid carrier or adhesive.

By way of further example, active ingredients are generally immersed within an excipient binder fluid, such as saline or an acetate composition, to make them injectible. Insulin, for example, is often placed in acetate mixes. Common pharmaceutical liquid carrier excipients include: water, distilled water, distilled water buffered, acetate, saline, phosphate, phosphate buffer with added protein, glycerin, saccharine, grapefruit aroma, methyl p-hydroxybenzoate, propyl p-hydroxybenzoate, sodium acetate, fructose, glucose or sucrose, hydrogenated glucose syrup, mannitol, maltitol, sorbitol, xylitol, gluten, tartrazine, arachis (peanut) oil, sesame oil, beeswax, benzyl alcohol, butylated hydroxyanisole, butylated hydroxytoluene, cetostearyl alcohol (including cetyl and stearyl alcohol), chlorocresol, edetic acid (edta), ethylenediamine, fragrances, hydroxybenzoates (parabens), imidurea, isopropyl palmitate, n-(3-chloroallyl)hexaminium chloride (quaternium 15), polysorbates, propylene glycol, sodium metabisulphite, wool fat and related substances including lanolin, and metacrystal solution.

Excipients typically include substituent components. For example, excipients may typically include one or more preservatives, like zinc, and one or more buffers, like dibasic sodium phosphate. Part of the present invention lies in the observation that certain pharmaceutical preparations contain liquid carrier excipients, and/or excipient ingredients, that inhibit the mobility, and hence deliverability of the active ingredient(s) responsively to insonification.

Referring now to FIG. 2, it shows a chart 200 indicative of human patient transdermal delivery rates for two commercially available insulin types responsively to insonification. The data of FIG. 2 is indicative of a transdermal delivery of u100 Humulin® and Humalog® to a human patient using a 125 mW, 30 kHz ultrasonic signal from a square, four-transducer array. Applicants have discovered that Humulin® is delivered at a greater rate than Humalog®.

Table 1 illustrates basic ingredients of Humulin® and Humalog®.

TABLE 1

| INGREDIENT | HUMULIN | | HUMALOG | |
|---|---|---|---|---|
| Biosynthetic human insulin | 100 | units/ml | 100 | units/ml |
| Glycerin | 16 | mg | 16 | mg |
| M-Cresol Preservative | 2.5 | mg | 3.15 | mg |
| Zinc Oxide | 0.017 | mg | 0.0197 | |
| Sodium Hydroxide + | Ph adjusted to | | Ph adjusted to | |
| Hydrochloric acid buffer | 7.0 to 7.8 | | 7.0 to 7.8 | |
| solution | | | | |
| Dibasic Sodium Phosphate | 0 | | 1.88 | mg |

The presence of dibasic sodium phosphate is a difference between the Humalog® and Humulin® types of insulin discovered. While not limiting the present invention, it is surmised that dibasic sodium phosphate is used as a buffering agent. Dibasic sodium phosphate has a specific gravity of 1.67 (higher than that of water, which is the bulk medium in both Humulin® and Humalog®). It is surmised this is the influencing factor that slows the delivery rate of Humalog® responsively to ultrasound.

By way of further, non-limiting example, ultrasound transduction through denser mediums is slower than through leaner or less dense materials. Ultrasound is reflected by dense materials and delivery power is impeded. It is surmised that the presence of high specific gravity dibasic sodium phosphate in Humalog® results in the impedance of ultrasound—thereby slowing transdermal delivery thereof.

These findings are corroborated by Table-2, which indicates that passive (control), e.g., non-ultrasound induced, delivery rates for Humalog® are greater than that for Humulin®. It is surmised that the presence of denser dibasic sodium phosphate in Humalog® enables it to “elute” or diffuse through the skin faster than Humulin®, which lacks the same.

TABLE 2

| Insulin Units | Humulin ®R | Humalog ® |
|---|---|---|
| Control | 4.1 ± 0.5 (n = 3) | 7.0 ± 4.4 (n = 5) |
| Single element cymbal | 7.4 ± 3.1 (n = 2) | (not performed) |
| Stack Array | 20.3 ± 9.3* (n = 3) | 19.9 ± 14.4* (n = 2) |
| Standard Array | 45.9 ± 12.9** (n = 15) | 30.8 ± 12.6** (n = 6) |

Basically, insonification changes the delivery equation of Humulin® and Humalog® as compared to passive configurations.

Further, dibasic sodium phosphate is a source for sodium ions in Humalog®. Sodium ions are positively charged. The presence of excess positive charges may lead to interactions with oppositely charged materials in a substance containing patch—and adsorb or absorb therein or thereto via weak electrostatic attraction. This may obstruct the path of the active ingredient, e.g., insulin, molecules through the solution from the patch to the skin, and there-through. Alternatively, excess positive charges may interact with substance component, e.g., insulin, molecules themselves, which then adsorb or absorb onto patch materials, or even the biotransformable layers of the skin, thereby slowing their delivery into viable skin or blood circulation.

Regardless, ultrasonically induced transdermal delivery of an insulin and dibasic sodium phosphate containing substance from a patch that may incorporate an absorbent material, may be hastened by reducing the relative amount of dibasic sodium phosphate therein. It is surmised that ultrasonically induced transdermal delivery of other substances, such as other pharmaceutical preparations including other active ingredients, can be similarly improved by reducing the amount of dibasic sodium phosphate therein. Similarly, it is surmised that ultrasonically induced transdermal delivery of substances, such as insulin containing drugs, can be improved by reducing the amount of excipient ingredients having a specific gravity greater than that of water. Similarly, it is also surmised that ultrasonically induced transdermal delivery of substances, such as insulin containing drugs, can be improved by predominantly using excipient ingredients having a specific gravity less than 1.67 and reducing those having specific gravities greater than 1.67.

By way of further, non-limiting explanation, the general equation employed to correlate ultrasound propagation speed with density of a material is

$${C}^{2} = \frac{K}{ρ} ,$$

where C=speed of ultrasound propagation, K=elastic modulus of the material and p=density of the material. Thus, the speed of ultrasound propagation is inversely relational to the density of a material. Moreover, acoustic impedance is directly proportional to the density of a material through which ultrasound propagates, and is given by Z=p×C, where Z=acoustic impedance, p=density of the material and C=speed of ultrasound propagation.

Table-3 also illustrates that the impedance to ultrasound propagation increases with higher density.

TABLE 3
Acoustic impedance

| | Z kg m⁻²s⁻¹ |
|---|---|
| Fat | 1.38 × 10⁶ |
| Muscle | 1.7 × 10⁶ |
| Air | 394.35 |
| Bone | 7 × 10⁶ |

As can be seen from Table-3, the impedance (Z) through materials is greater for denser materials (e.g., bone has the highest density among the four material listed in the table and fat has the lowest). Thus, the higher specific gravity dibasic sodium phosphate acting as the major influencing factor for slower delivery of Humalog® under the effect of ultrasound is supported by the empirical relations stated above.

It should be understood that just as formulations are made using conventional processes according to conventional formulae, re-formulated formulations, such as drugs and substances, that have a reduced amount of dibasic sodium phosphate, a reduced amount of excipient ingredients having a specific gravity greater than that of water, and/or predominantly use excipient ingredients having a specific gravity less than 1.67 may be similarly made using re-formulated formulae.

According to an aspect of the present invention, substances can be identified as good candidates for ultrasonic transdermal delivery or re-formulation for transdermal delivery using similar criteria. For example, a list of substances and corresponding compositions may be made or acquired. Such a list may be recorded in a computer readable media, for example. Those substances having excipient ingredients with specific gravities under 1.67 and/or omitting dibasic sodium phosphate and/or having excipient components with specific gravities around that of water may be selected as good candidates for ultrasonically induced transdermal delivery. Similarly, those substances having excipient ingredients with specific gravities around 1.67 or higher, and/or including dibasic sodium phosphate may be selected as less attractive candidates for reformulation for ultrasonically induced transdermal delivery. The good candidates are predicted to generally have a higher ultrasonically induced transdermal deliver rate of the active ingredient(s) thereof higher than less attractive candidates have the same active ingredient(s). By way of further, non-limiting example only, Humulin® may be classified in a first class as being a good candidate, while Humalog® may be classified in a second class as being a less attractive candidate for rapid transdermal delivery from a patch responsively to ultrasound insonification. Such a classifying may be carried out by a general purpose computing device having access to the computer readable media containing the list of substances.

It will be apparent to those skilled in the art that modifications and variations may be made in the apparatus and process of the present invention without departing from the spirit or scope of the invention. It is intended that the present invention cover the modification and variations of this invention provided they come within the scope of the appended claims and their equivalents.

What is claimed is:

1. A method for improving transdermal delivery of an active ingredient in a formulation made in accordance with a dibasic sodium phosphate containing formula responsively to insonification thereof, comprising:
reducing the amount of dibasic sodium phosphate in the formula to provide a reduced dibasic sodium phosphate formula; and,
making a formulation in accordance with the reduced dibasic sodium phosphate formula.

2. The method of claim 1, wherein said substance further comprises insulin.

3. The method of claim 2, wherein said insonifying comprises ultrasonic insonification.

4. The method of claim 1, wherein said reducing comprises replacing said dibasic sodium phosphate with a component having a specific gravity less than 1.67.

5. The method of claim 1, wherein said reducing comprises replacing said dibasic sodium phosphate with a component having a specific gravity approximately that of water.

6. A method for improving transdermal delivery of an active ingredient in a formulation made in accordance with an excipient containing formula, wherein said excipient includes at least one component having a specific gravity of at least around 1.67, responsively to insonification thereof, comprising:
reducing the amount of excipient component having a specific gravity of at least around 1.67 to provide a second formula; and,
making a formulation in accordance with the second formula.

7. The method of claim 6, wherein said at least one active ingredient comprises insulin.

8. The method of claim 7, wherein said insonification comprises ultrasonic insonification.

9. The method of claim 7, wherein said reducing comprises replacing said excipient component having a specific gravity of at least around 1.67 with a component having a specific gravity less than 1.67.

10. The method of claim 7, wherein said reducing comprises replacing said excipient component having a specific gravity of at least around 1.67 with a component having a specific gravity approximately that of water.

11. A method for classifying the predicted suitability of a substance for ultrasonically induced transdermal delivery into a patient comprising:
determining if the substance contains dibasic sodium phosphate; and,
if it does, classifying the substance in a first class; and,
if it does not, classifying the substance in a second class;
wherein, said first class is associated with substances having a relatively high predicted transdermal delivery rate, and said second class is associated with substances having a relatively low predicted transdermal delivery rate.

12. The method of claim 11, further comprising providing a list of substances, wherein said determining and classifying occurs for each of said substances on said list.

13. A method for classifying the predicted suitability of a substance for ultrasonically induced transdermal delivery into a patient comprising:
determining if the substance contains an excipient component having a specific gravity of at least around 1.67; and,
if it does, classifying the substance in a first class; and,
if it does not, classifying the substance in a second class;
wherein, said first class is associated with substances having a relatively high predicted transdermal delivery rate, and said second class is associated with substances having a relatively low predicted transdermal delivery rate.

14. The method of claim 13, further comprising providing a list of substances, wherein said determining and classifying occurs for each of said substances on said list.
